(19) United States
(12) Patent Application Publication
(10) Pub. No.: US 2026/0347768 A1
(43) Pub. Date: Nov. 5, 2026

(54) DISEASE MARKERS OF EARLY AGE-RELATED MACULAR DEGENERATION USING A NOVEL BRUCH'S MEMBRANE MODE

(71) Applicants: Elizabeth Vargis, Logan, UT (US); Justin A. Jones, Nibley, UT (US); Dillon Weatherston, Logan, UT (US); Emilee Rickabaugh, Shelley, ID (US)

(72) Inventors: Elizabeth Vargis, Logan, UT (US); Justin A. Jones, Nibley, UT (US); Dillon Weatherston, Logan, UT (US); Emilee Rickabaugh, Shelley, ID (US)

(73) Assignee: Utah State University, Logan, UT (US)

(21) Appl. No.: 19/666,020

(22) Filed: May 1, 2026

Related U.S. Application Data
(60) Provisional application No. 63/798,463, filed on May 1, 2025.

(51) Int. Cl. C12N 5/079

(52) U.S. Cl. CPC C12N 5/0621; C12N 2503/02; C12N 2503/04; C12N 2533/50

(57) ABSTRACT

The present disclosure highlights the use of the physical properties of αHM to produce compositions for the modeling of disease including AMD. Thus, described herein is compositions and systems comprising αHM and methods of using the compositions described herein for the modeling of disease.

### RELATED APPLICATIONS

This application claims priority to U.S. Provisional Application No. 63/798,463 filed on May 1, 2025 which is incorporated by reference in its entirety.

### FEDERALLY SPONSORED RESEARCH

This invention was made with government support under Grant Number 1R15EY028732-01A1 awarded by the National Institutes of Health. The government has certain rights in the invention.

### FIELD

The present disclosure highlights the use of the physical properties of a hagfish membrane (αHM) to produce compositions for the modeling of disease including AMD, diabetic retinopathy, retinitis pigmentosa, Stargardt disease, and glaucoma. Thus, described herein are compositions and systems comprising αHM and methods of using the compositions described herein for the modeling of disease.

### BACKGROUND

Bruch's membrane is a proteinaceous membrane that separates the retinal pigment epithelium (RPE) from the choroid, or vasculature of the subretina. The RPE serves as a caretaking layer for photoreceptors, providing nutrients and disposing of waste products. Together with Bruch's membrane, the RPE regulates passage of materials through the retina and makes up the outer blood-retinal barrier (oBRB). Passage of materials through the oBRB is largely controlled by RPE expression of junctional proteins, such as zonula occludens-1, that maintain a tight monolayer (O'Leary F, et al. FEBS J. 290:878-91 (2023) and Shu D Y, et al. Int J Mol Sci. 21:4271 (2020)). RPE cells also act as caretakers for photoreceptors, digesting outer segments and providing key nutrients for the visual cycle (Strauss O, The retinal pigment epithelium. University of Utah Health Sciences Center 1995). During aging, Bruch's membrane thickens, stiffens, and becomes less permeable, which can inhibit its function in regulating transport in the subretinal tissue. These changes are implicated in the development of age-related macular degeneration (AMD), which is a leading cause of blindness in elderly populations. By 2040, it is expected that over 288 million people will be diagnosed with AMD worldwide. Unfortunately, there is no cure for AMD, and the majority of treatments are administered after vision loss has already occurred. (See Mitchell P, et al. Lancet. 392:1147-59 (2018) and Chaudhuri M, et. al. Cureus. 2023). The lack of available treatments is, in part, due to a lack of information about the development and progression of AMD. Therefore, a better understanding of early AMD is essential for developing novel treatments and improving vision outcomes in patients.

Age-related macular degeneration is characterized by degradation to the RPE layer, which, in turn, affects photoreceptor function and vision. Early age-related macular degeneration typically involves the buildup of lipids and proteins between the RPE and Bruch's membrane called drusen in what is known as dry AMD. Specifically, materials known as basal laminar deposits (BLamDs) and basal linear deposits (BLinDs) build up beneath the RPE. (See Sura et al. Invest Ophthalmol Vis Sci. 61:19 (2020) and Chen, et. al. Invest Ophthalmol Vis Sci. 62:33 (2021). As BLamDs and BLinDs increase the thickness of Bruch's membrane, their rate of accumulation increases, culminating in domed deposits called drusen between the RPE and Bruch's membrane. (See Curcio C A, et. al. J Lipid Res. 51:451-67 (2010) and Wang L, et al. PLoS ONE. 5:e10329 (2010). Drusen formation beneath the RPE is correlated with subsequent fluid accumulation, both of which cause low levels of stress and are implicated in cell damage and the eventual development of AMD [Booij J C, et. al. Prog Retin Eye Res. 29:1-18 (2010); Bonilha V L. Clin Ophthalmol Auckl N Z. 2:413-24 (2008); Zhang X, et. al. Eye. 35:121-33 (2021); and Sikorski B L, et. al. Ophthalmology. 118:82-92 (2011)). While drusen formation is a normal part of aging, higher numbers of larger drusen deposits, which in turn cause more mechanical stress, are correlated with an increased risk and quicker progression of AMD. (See Paterson C, et al. Lab Chip. 24:5338-46 (2024) and Farjood F, et. al. J Biol Eng. 14:13 (2020)). One key component of drusen is apolipoprotein E (ApoE), which normally aids in cholesterol transport. Isoforms of ApoE contribute to age-related macular degeneration and other age-related diseases such as Alzheimer's disease (AD). (See Mak A C Y, et al. JAMA Neurol. 71:1228-36 (2014); Bojanowski C M, et al. Environ Mol Mutagen. 47:594-602 (2006); and Toops K A, et. al. A. Apolipoprotein E isoforms and AMD. Retin Degener Dis, Springer International pp. 3-9 (2016)). The impact of ApoE on AD development has been extensively studied, and its role in AD pathology may offer parallel insights as to how it affects AMD progression. (See Huang Y, et al. Neurobiol Dis. 2014). For example in AD, ApoE overexpression decreases the integrity of the blood-brain barrier as marked by a decrease in junctional protein expression (see Liao F, et al. J Neurosci. 34:7281-92 (2014) and Donahue J E, et. al. J Neuropathol Exp Neurol. 67:261-70 (2008)). The link between ApoE and barrier function in the retina during AMD, however, is less clear. Decreased retinal barrier integrity and ApoE overexpression are both associated with AMD, making them ideal targets for studies modeling early AMD.

Drusen development is often followed by regions of RPE death known as geographic atrophy (GA). In some cases, stress to the RPE causes an overexpression of pro-angiogenic factors such as vascular endothelial growth factor (VEGF) that cause choroidal cells to invade the RPE through Bruch's membrane in choroidal neovascularization (CNV), or wet AMD. These new choroidal cells often leak and cause additional stress to the RPE, exacerbating the rate of cell death and blindness, leading to increased angiogenic factor expression in a positive feedback loop. Interestingly, the majority of available treatments for age-related macular degeneration only target choroidal neovascularization, which occurs in just 10% of cases. The lack of earlier treatments is largely due to a lack of understanding of how early age-related macular degeneration progresses.

A likely contributor to age-related macular degeneration is the thickening of Bruch's membrane. Early age-related macular degeneration is characterized by drusen formation, which could be caused by decreased diffusion rates through an aged Bruch's membrane. Additionally, previous work has shown that in early stages of age-related macular degeneration, matrix metalloproteases (MMPs) and tissue inhibitors of metalloprotease (TIMPs) are dysregulated. MMPs and TIMPs play a key role in regulating the RPE's ECM, which makes up part of Bruch's membrane. The link between age-related macular degeneration and dysregulation of Bruch's membrane further indicates that Bruch's membrane plays a role in disease progression.

Unfortunately, the direct impact of changes to Bruch's membrane on other layers of the subretina is difficult to assess in vivo due to the inaccessibility of these layers. Determining the impact of changes to Bruch's membrane on RPE health in vitro is also complicated due to the lack of tunable models of Bruch's membrane. Transwell cell culture inserts are a common platform for retinal research, but they are porous, made of plastic materials such as polycarbonate, polyester, and polytetrafluoroethylene, and only manufactured at specific thicknesses, meaning they cannot be tuned to match changes to Bruch's membrane. Other models of Bruch's membrane such as electrospun scaffolds, spider silk membranes, and silk fibroin membranes match in vivo properties of a healthy Bruch's membrane but have not been tuned to mimic the aging process.

One novel model of Bruch's membrane that shows promise in mimicking age-related changes to Bruch's membrane is the a hagfish membrane (αHM). Previous work has shown that αHM not only matches in vivo properties of a healthy Bruch's membrane, but it can also be tuned to match physical and mechanical changes to Bruch's membrane with age. Therefore, there still exists a need for a tunable platform to assess the direct impact of changes in Bruch's membrane on RPE health and the development of AMD.

### BRIEF SUMMARY OF THE DISCLOSURE

One embodiment described herein is a composition comprising: (a) a membrane for supporting growth of cells, the membrane being made of hagfish intermediate filament α wherein the membrane has a minimum thickness of 12 μm; and (b) a layer of retinal pigment epithelial (RPE) cells, or retinal pigment epithelial cell precursors, on the membrane.

In some embodiments, the RPE cells are primary RPE cells. In some embodiments the RPE cells are a monolayer. In some embodiments, the layer of RPE cells is at least a density of 3×10⁵ cells/cm².

One embodiment described herein is a synthetic membrane system, comprising: a membrane having: a side facilitating the growth of a monolayer of cells; a thickness greater than 12 μm; and the membrane comprising hagfish intermediate filament α.

In some embodiments, the system further comprises: a cell culture insert on which the membrane sits; and a silicone ring holding the membrane to the cell culture insert.

In some embodiments, the membrane is circular with a diameter of between about 10 mm to about 20 mm.

One embodiment described herein is a method of making a Bruch's membrane model comprising: (i) spreading a solution of hagfish intermediate filament α onto a disk to form a membrane at a concentration of 1% w/v of hagfish intermediate filament α; (ii) drying the membrane; (iii) removing the dried membrane from the disk; (iv) rehydrating the membrane; (v) adding the dried membrane to a cell culture insert; and (vi) seeding the membrane-cell culture insert with cells.

In some embodiments, the method further comprises: (vii) growing the cells in a monolayer.

In some embodiments, the cells are retinal pigment epithelial (RPE) cells. In some embodiments, the RPE cells are primary porcine RPE cells. In some embodiments, the RPE cells are seeded at a density of 3×10⁵ cells/cm².

On embodiment described herein includes a synthetic membrane system made by the methods described herein.

One embodiment described herein is a method of identifying compositions for treating or lessening the severity of age-related macular degeneration in a subject comprising: (i) administering a test composition to the Bruch's membrane model made by the method disclosed herein; and (ii) measuring the effect on the Bruch's membrane model; wherein the composition is selected as a candidate on the basis of treating or lessening the severity of age-related macular degeneration.

In some embodiments, the treating or lessening the severity of age-related macular degeneration comprises decreasing the thickness, decreasing the stiffness, increasing the permeability, or increasing the elasticity of the Bruch's membrane.

### BRIEF DESCRIPTION OF THE FIGURES

FIG. 1 shows that thickened membranes lead to an increase in RPE cell death. Between 3 and 5 weeks, RPE cell death begins to occur on UtαHM (A) compared to healthy RPE monolayer on αHM (B). RPE cells form domes on TαHM after 2 weeks of culture (out of focus, C, and in focus, D, circled), scale=100 μm.

FIG. 2 is a plot of cells cultured on UTαHM exhibit reduced viability compared to cells on other membrane types. Error bars represent standard deviation, n=3, p<0.5. Asterisks represent groups that were statistically different from each of the other three treatment groups as determined by a one-way ANOVA with post hoc analysis

FIG. 3 shows deposits formed on thickened membranes exhibit high lipid content. Lipid stain (left) on sub-RPE deposit found after cells had detached from a TαHM and a Brightfield image of the deposit (right) scale=100 μm.

FIG. 4 shows the ICC images on all membrane types after 21 days of pRPE culture. Cells growing on αHMs have healthy morphology (A) and consistent levels of ZO-1 and ApoE expression (B and C). Cells growing on TαHMs have gaps in their monolayer (E, arrow), which is then seen as a disruption in ZO-1 (F, arrow). The Brightfield image of cells growing on UTαHMs shows a more consistent monolayer (I), but a disruption of ZO-1 expression (K, arrows) adjacent to an area of increased ApoE expression (J, arrow). Transwell cultures exhibit normal morphology (M) and localized ApoE (O, arrow), but no corresponding disruption in ZO-1 formation across the culture (N), scale bars=50 μm.

FIG. 5 shows in A) the TEER values of pRPE cells on all membrane types between 7 and 21 days of cell culture. Asterisks represent statistically significant differences between readings. n=3. B) shows the percent change in TEER reading from the time cells reached 300 Ω*cm² until the end of the 5-week culture period, n=6.

FIG. 6 is ELISA data showing angiogenic proteins of interest after 14, 17, and 21 days of cell culture. The y axis for basal VEGF is in log scale for ease of viewing, and basal samples were collected from both medium in the basal chamber and from the hagfish membrane. Error bars are standard deviation, asterisks represent statistical significance, p<0.05, n=3.

### DETAILED DESCRIPTION OF THE DISCLOSURE

The present disclosure highlights the use of the physical properties of αHM to produce compositions for the modeling of disease including AMD, diabetic retinopathy, retinitis pigmentosa, Stargardt disease and glaucoma. Thus, described herein is compositions and systems comprising αHM and methods of using the compositions described herein for the modeling of ocular tissue diseases.

In some embodiments, the present disclosure relates to porcine retinal pigment epithelium (RPE) cells as a mimetic RPE layer, and hagfish membranes as a tunable Bruch's membrane. In some embodiments, the hagfish membrane was created in various thicknesses to represent to represent healthy, aged, and extreme (diseased) states. The healthy membrane model is of normal thickness (αHM), the aged membrane model is of increased thickness (TαHM), and the extreme (diseased) state membrane model is the thickest (UTαHM).

### DEFINITIONS

The following definitions are meant to clarify, but not limit, the terms defined. If a particular term used herein is not specifically defined, such term should not be considered indefinite. Rather, terms are used within their accepted meanings.

As used herein, “retinal pigment epithelial cells” or “RPE cells” refer to cells of the retinal pigment epithelium. This epithelium is a monolayer of pigmented cells situated between the neuroretina and the choroid The RPE is of neuroectodermal origin and is therefore considered to be part of the retina. The apical membrane of the RPE faces the photoreceptor's outer segments and its basolateral membrane faces Bruch's membrane, which separates the RPE from the fenestrated endothelium of the choriocapillaris. The RPE constitutes the “outer blood-retinal barrier” (BRB or oBRB). The inner BRB is mainly constituted by endothelial cells. Tight junctions between neighboring RPE cells and neighboring endothelial cells are essential in the strict control of fluids and solutes that cross the BRB as well as in preventing the entrance of toxic molecules and plasma components into the retina.

As used herein, the terms “age-related macular degeneration” or “AMD” or “ARMD” refer to an eye disease with its onset usually after age 60 that can progressively destroy the macula, the central portion of the retina, impairing central vision. Age-related macular degeneration rarely causes blindness because only the center of vision is affected. Injury to the macula in the center of the retina, however, can impair the ability to see straight ahead clearly and make it difficult to read, drive, or perform other daily activities that require fine central vision. The macula is in the center of the retina at the back of the eye. During activities such as reading, the light is focused onto the macula where millions of cells change the light into nerve signals that travel to the brain and tell it what the patient is seeing (central vision). With normal central vision, the ability to read, drive, and perform other activities that require fine, sharp, straight-ahead vision is kept.

“Treating” or “treatment” as used herein refers to the treating or treatment of a disease or medical condition (such as age-related macular degeneration) in a patient, such as a mammal (particularly a human) which includes curing, reversing, alleviating, reducing the severity of, inhibiting the progression of, or reducing the likelihood of a disease, disorder, or condition or one or more symptoms or manifestations of a disease, disorder or condition. Because a loss of the normal RPE layer and Bruch's membrane results in the development of AMD, a change in membrane thickness and stiffness, and a change in elasticity and permeability, would facilitate progress towards a desirable state in individuals exhibiting a need for treatment of AMID or related disorders. Any approach that neutralizes the thickening, stiffening, and decrease in permeability or elasticity of the Bruch's membrane can be used to effectuate amelioration of AMD or related disorders. Similarly, the modeling of other diseases resulting in changes to the Bruch's membrane is possible including but not limited to diabetic retinopathy, retinitis pigmentosa, Stargardt disease and glaucoma could also be modeled.

As used herein, the term “cell culture insert” refers to a cell culture insert that is generally used for cell culture and is inserted into each well of a dish or a multi-well plate. A typical insert is a cylindrical container having a size that can be inserted into a dish or well and has a bottom and a side to ensure a certain volume that allows cell culture. According to the present disclosure, a cell culture insert is provided by disposing the standard porous membrane at the bottom of a cylindrical container and replacement using the methods disclosed herein. Here, the cylindrical container has a space that allows cell culture inside the cell culture insert and has a size that can be inserted into a typical dish or well. In order not to contact the bottom of the dish or well, the upper surface of the cylindrical container has an arm structure that can be hung on the upper surface of the dish or well. The cylindrical container is not limited as long as it has the above-mentioned shape, and a commonly used, general-purpose, or commercially available container can be used, and a cell culture insert is provided by removing the standard porous membrane to the bottom surface of this cylindrical container. Alternatively, a commonly used, general-purpose, or commercially available insert in which a membrane is disposed may be used to apply a membrane in the present disclosure. The size and shape of the membrane used in the present disclosure can be easily molded to match the shape of the bottom of the insert to be used. The bottom of the insert generally has a circular or elliptical shape, and if it is circular, it is equal to or smaller than the upper opening diameter of the dish or well. Therefore, the diameter of the membrane disposed at the bottom is appropriately adjusted to be equal to or smaller than the opening diameter of the dish (e.g., nominal diameter 5 mm, nominal diameter 10 mm, nominal diameter 15 mm, nominal diameter 25 mm, nominal diameter 35 mm, nominal diameter 60 mm, nominal diameter 100 mm, nominal diameter 150 mm, etc.) or well (e.g., 6 wells, 12 wells, 24 wells, 96 wells, etc.) into which the cell culture insert is inserted and used. The nominal diameter of the dish is a general one and does not represent the exact diameter. For example, the diameter of the membrane is typically 5 to 20 mm and that is the size that fits into the dish or well.

The insert used is preferably sterilized. The sterilization treatment is not particularly limited, and any sterilization treatment can be selected from dry heat sterilization, ethylene oxide gas sterilization, high-pressure steam sterilization, sterilization with a disinfectant such as ethanol, and sterilization with electromagnetic waves such as ultraviolet rays and gamma rays and/or radiation. A plurality of these sterilization methods can also be combined and used appropriately.

### Membrane Compositions and Systems

Understanding how aging in the Bruch's membrane affects the development of AMD benefits from a tunable model that can mimic the aging process. The present disclosure comprises primary porcine RPE (pRPE) cells as a mimetic RPE layer, and hagfish membranes as a tunable Bruch's membrane. In some embodiments, the hagfish membrane model, was cultured with pRPE cells on an a hagfish membrane (αHM), thickened αHM (TαHM), and ultra-thickened αHM (UTαHM) to represent healthy, aged, and extreme states, respectively.

In some embodiments, the present disclosure relates to a composition comprising: a membrane for supporting growth of cells, the membrane being made of hagfish intermediate filament α wherein the membrane has a minimum thickness of 12 μm; and a layer of retinal pigment epithelial (RPE) cells, or retinal pigment epithelial cell precursors, on the membrane.

In some embodiments, the membrane has a thickness of 12 μm. In some embodiments, the membrane has a thickness of greater than 12 μm. In some embodiments, the membrane has a thickness of about 15 μm, about 16 μm, about 17 μm, about 18 μm, about 19 μm, about 20 μm, about 21 μm, about 22 μm, about 23 μm, about 24 μm, or about 25 μm.

The compositions, systems, and methods disclosed herein utilize retinal pigment epithelial (RPE) cells and endothelial cells. The cells can be from any mammal. The cells can be from a human, a non-human primate, or any other mammalian subject, such as a canine, porcine, feline, bovine, equine, or rodent subject. In a specific non-limiting example, the cells used in the methods disclosed herein, including the RPE cells and endothelial cells, are human. Retinal pigment epithelial (RPE) cells can be characterized based upon their pigmentation, epithelial morphology, and apical-basal polarity.

In some embodiments, the RPE cells are primary porcine RPE cells. In some embodiments, the layer of RPE cells is a monolayer. In some embodiments, the layer of RPE cells is a monolayer with a dome. In some embodiments, the layer of RPE cells is a monolayer with drusen. In some embodiments, the layer of RPE cells is at least a density of 3×10⁵ cells/cm².

In some embodiments, cells were cultured for up to three weeks. In some embodiments, cells were cultured for up to one month. In some embodiments, cells were cultured for up to two months. In some embodiments, cells were cultured for up to three months. In some embodiments, cells were cultured for up to four months. In some embodiments, cells were cultured for up to five months. In some embodiments, cells were cultured for up to six months. In some embodiments, cells were cultured for up to seven months. In some embodiments, cells were cultured for up to eight months. In some embodiments, cells were cultured for up to nine months. In some embodiments, cells were cultured for up to ten months. In some embodiments, cells were cultured for up to eleven months. In some embodiments, cells were cultured for up to one year.

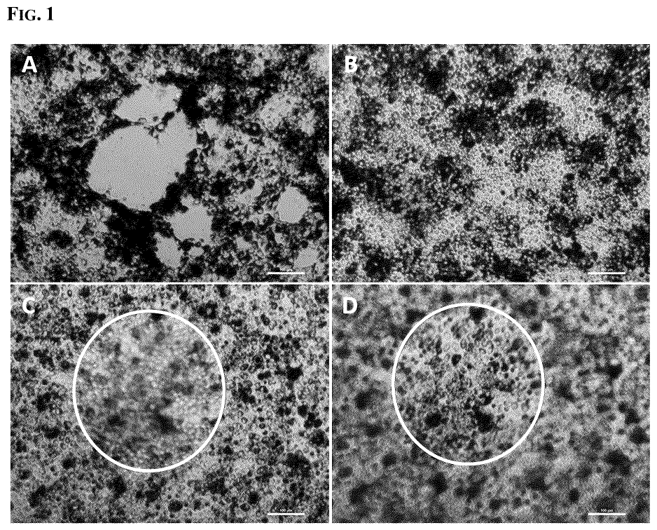

In some embodiments, the cells maintained the typical pigmentation and cobblestone morphology found in RPE cells. In some embodiments, the cells formed domes in their monolayer (FIG. 1).

In some embodiments, the membrane has decreased permeability and increased thickness compared to a healthy membrane. In some embodiments, the RPE monolayer has domes and deposit formation. In some embodiments, the membrane has decreased permeability and increased thickness compared to a healthy membrane and the RPE monolayer has domes and deposit formation.

In some embodiments, the disclosure relates to a synthetic membrane system, comprising: a membrane having: a side facilitating the growth of a monolayer of cells; a thickness greater than 12 μm; and the membrane comprising hagfish intermediate filament α.

In some embodiments, the synthetic membrane system further comprises a cell culture insert on which the membrane sits and a silicone ring holding the membrane to the cell culture insert.

In some embodiments, the membrane is circular with a diameter of between about 10 mm to about 20 mm. In some embodiments, the diameter is about 10 mm, about 12 mm, about 14 mm, about 15 mm, about 16 mm, about 17 mm, about 18 mm, about 20 mm.

In some embodiments, the disclosure relates to a synthetic membrane system, comprising: a membrane having: a side facilitating the growth of a monolayer of cells; a greater than 1% protein concentration and the membrane comprising hagfish intermediate filament α.

In some embodiments, the disclosure relates to the composition or synthetic membrane system of the disclosure as an in vitro model of the aging Bruch's membrane. In some embodiments, the disclosure relates to the composition or synthetic membrane system of the disclosure as an in vitro model of an AMD Bruch's membrane. In some embodiments, the disclosure relates to the composition or synthetic membrane system of the disclosure as an in vitro model of a severe AMD Bruch's membrane. In some embodiments, the disclosure relates to the composition or synthetic membrane system of the disclosure as an in vitro model of diabetic retinopathy. In some embodiments, the disclosure relates to the composition or synthetic membrane system of the disclosure as an in vitro model of retinitis pigmentosa. In some embodiments, the disclosure relates to the composition or synthetic membrane system of the disclosure as an in vitro model of Stargardt disease. In some embodiments, the disclosure relates to the composition or synthetic membrane system of the disclosure as an in vitro model of glaucoma.

In some embodiments, the composition or synthetic membrane system of the disclosure additionally includes the formation of drusen deposits. In some embodiments, the composition or synthetic membrane system of the disclosure additionally includes sub-RPE deposits.

### Use of Membrane Compositions and Systems

One embodiment of this disclosure includes a method of producing a Bruch's membrane model comprising: spreading a solution of hagfish intermediate filament α onto a disk to form a membrane at a concentration of 1% w/v of hagfish intermediate filament α; drying the membrane; removing the dried membrane from the disk; rehydrating the membrane; adding the dried membrane to a cell culture insert; and seeding the membrane-cell culture insert with cells.

In some embodiments, the method further comprises growing the cells in a monolayer.

In some embodiments, the methods disclosed herein utilize retinal pigment epithelial (RPE) cells and endothelial cells. The cells can be from any mammal. The cells can be from a human, a non-human primate, or any other mammalian subject, such as a canine, porcine, feline, bovine, equine, or rodent subject. In a specific non-limiting example, the cells used in the methods disclosed herein, including the RPE cells and endothelial cells, are human. Retinal pigment epithelial (RPE) cells can be characterized based upon their pigmentation, epithelial morphology, and apical-basal polarity.

In some embodiments, the method utilizes RPE cells that are primary porcine RPE cells. In some embodiments, the layer of RPE cells is a monolayer. In some embodiments, the layer of RPE cells is a monolayer with a dome. In some embodiments, the layer of RPE cells is a monolayer with drusen. In some embodiments, the layer of RPE cells is at least a density of 3×10⁵ cells/cm². In some embodiments, the layer of RPE cells is a density of less than 3×10⁵ cells/cm².

In some embodiments, the method has a membrane with a thickness of 12 μm. In some embodiments, the membrane has a thickness of greater than 12 μm. In some embodiments, the membrane has a thickness of about 15 μm, about 16 μm, about 17 μm, about 18 μm, about 19 μm, about 20 μm, about 21 μm, about 22 μm, about 23 μm, about 24 μm, or about 25 μm.

In some embodiments, the disclosure relates to a synthetic membrane system made by any methods disclosed herein.

In some embodiments, the present disclosure relates to a method for establishing a model. In some embodiments, disclosed herein is a method for screening a drug and/or composition. In some embodiments, the present disclosure relates to use of a membrane in establishing a model and in screening a drug and/or a composition.

In some embodiments the disclosure relates to a method of identifying compositions for treating or lessening the severity of disease in a subject comprising: administering a test composition to the Bruch's membrane model made by any of the method of the disclosure; and measuring the effect on the Bruch's membrane model; wherein the composition is selected as a candidate on the basis of treating or lessening the severity of disease.

In some embodiments, the disease is age-related macular degeneration. In some embodiments, the disease is diabetic retinopathy. In some embodiments, the disease is diabetic retinopathy. In some embodiments, the disease is retinitis pigmentosa. In some embodiments, the disease is Stargardt disease. In some embodiments, the disease is Glaucoma.

In some embodiments the disclosure relates to a method of identifying compositions for treating or lessening the severity of age-related macular degeneration in a subject comprising: administering a test composition to the Bruch's membrane model made by any of the method of the disclosure; and measuring the effect on the Bruch's membrane model; wherein the composition is selected as a candidate on the basis of treating or lessening the severity of age-related macular degeneration.

In some embodiments, the treating or lessening the severity of age-related macular degeneration comprises decreasing the thickness, decreasing the stiffness, increasing the permeability, or increasing the elasticity of the Bruch's membrane. In some embodiments, the treating or lessening the severity of age-related macular degeneration comprises decreasing the thickness of the Bruch's membrane. In some embodiments, the treating or lessening the severity of age-related macular degeneration comprises decreasing the stiffness of the Bruch's membrane. In some embodiments, the treating or lessening the severity of age-related macular degeneration comprises increasing the permeability of the Bruch's membrane. In some embodiments, the treating or lessening the severity of age-related macular degeneration comprises increasing the elasticity of the Bruch's membrane.

In some embodiments, the treating or lessening the severity of age-related macular degeneration comprises 1) reducing a drusenoid lesion, e.g., on retinal tissues; 2) reducing drusen-like nodules, e.g., under the retinal pigment epithelium layer in the eye; 3) reducing pyroptosis of the retinal pigment epithelium cells in the eye; 4) reducing activation of the complement system and/or inflammation in the eye; 5) reducing secretion of active IL-1β and/or IL-18 by retinal pigment epithelium cells in the eye; or 6) any combination of 1)-5).

### EXAMPLES

### Example 1: Production of Hagfish Membranes

### Hagfish Membrane Preparation

To prepare hagfish membranes, lyophilized hagfish intermediate filament α was solubilized in 75% formic acid. Formulations of 0.5% w/v, 0.75% w/v, and 1% w/v were used to make three different membrane types: a hagfish membrane (αHM), thickened αHM (TαHM) and ultra-thickened αHM (UTαHM), respectively. The three protein-formic acid solutions were then spread onto ~42 mm diameter polydimethylsiloxane (PDMS) discs and dried for 24 hours. Dry hagfish membranes were then peeled from the PDMS, punched into 15.875 mm diameter discs and mounted on cell culture inserts using a previously described method (see Rickabaugh, E. et. al. ACS Biomat. Sci. Eng. 2023, 9(8), 5051). Sterilization and preparation for cell culture also followed a previously used protocol.

UTαHM were measured to compare the thickness to the previously defined αHM and TαHM. Samples were measured by cutting small ~2 mm-wide slices from the punched membranes and propping them vertically under a BA310 microscope (Motic). The slices were imaged at 400× magnification, and three measurements were taken on each slice and averaged.

Hagfish intermediate filament alpha was solubilized in formic acid at 0.5% w/v, 0.75% w/v, and 1% w/v concentrations to create a hagfish membrane (αHM), thickened αHM (TαHM), and ultra-thickened (UTαHM), respectively. The three membrane compositions were measured, and values are reported in Table 1.

TABLE 1
Compilation of various hagfish membrane thicknesses, along with protein
content, compared to in vivo values reported in the literature

| | Protein concentration | Thickness |
|---|---|---|
| Membrane Type | (% w/v) | (μm) |
| In vivo healthy Bruch's | N/A | 2-4* |
| membrane | | |
| In vivo aged Bruch's | N/A | 6-10* |
| membrane | | |
| aHM | 0.50 | 5.75 ± 1.10† |
| TaHM | 0.75 | 9.64 ± 1.95† |
| UTaHM | 1.00 | 14.62 ± 4.62 |

*(See Booij, Prog Retin Eye Res, 2010, 29: 1; Curcio C A, In: Retina, Elsevier, 2013: pp. 465-481; and Okubo A, Invest Ophthalmol Vis Sci. 1999; 40: 443-9)
†See Rickabaugh, E. et. al. ACS Biomat. Sci. Eng. 2023, 9(8), 505).

### Mechanical Testing.

Immediately upon drying, rHIF membranes were cut into 2 mm wide strips, stored overnight, and adhered to 8 mm wide plastic cards on either end of the membrane sample. The samples were then hydrated in DPBS −/− (Cytiva) and placed on a plastic frame with a testing gap of 3 mm. Prior to testing, membranes were treated to mimic the process used for seeding with cells. Membranes were treated by soaking in 50% ethanol (EtOH) for 15 min, washing in DPBS −/− for 2 min, soaking in 100% EtOH for 15 min, and then washing in DPBS −/− twice for 2 min each. A MicroTester G2 (CellScale) with a 0.5588 mm diameter tungsten rod with a beam length of 46-52 mm was then used to stretch the membranes to failure at a test speed of 25 μm/s. All tests used DPBS −/− in the testing bath. MicroTester data were then used to generate stress-strain curves. The initial linear portions of these curves were used to calculate the elastic moduli of the rHIF membranes.

The elastic moduli of rHIF membranes were quantified (Table 2). All three types of rHIF membranes had values between 2 and 5 MPa. The rHIF membranes also had statistically significant lower elastic moduli than Transwells (p<0.05, Table 2) as previously reported (see Rickabaugh, E. et. al. ACS Biomat. Sci. Eng. 2023, 9(8), 5051).

Normally, adding more protein should not change the modulus but surprisingly there's a marked increase in elastic modulus from αHM to TαHM and a decrease in modulus back to that of αHM when further thickened to UTαHM (Table 2).

TABLE 2
Elastic Modulus of Bruch's Membrane

| Membrane Type | Elastic Modulus (MPa) |
|---|---|
| aHM | 2.1 ± 0.53 |
| TaHM | 4.29 ± 0.72 |
| UTaHM | 1.95 ± 0.45 |
| PE Transwell (previously reported) | ~2000 |

### Permeability Assays.

To determine the permeability of the rHIF membranes, permeability assays were performed at 0 (without cells), 2, and 7 days of cell culture. A concentrated FITC-Dextran (Millipore Sigma) solution of one of three different molecular weights (10, 70) at 5 mg/mL was added to the apical side of the membrane. Samples were taken from the basal side of the membrane at 5, 30, 60, and 120 min and placed in a black opaque well plate. The fluorescent readings of the basal samples were then quantified using a fluorometer and translated to FITC-Dextran concentration using a calibration curve. From this change in concentration, a molecular flux was calculated to determine permeability coefficients using equation 1.

$${P}_{app} = \frac{dQ}{dt} \times \frac{1}{{A}_{m} {C}_{a}} \qquad ( 1 )$$

In equation 1, the permeability coefficient (cm/s) was calculated by multiplying the molecular flux (mg/s) by the inverse of the product of the membrane surface area (cm²) and the concentration of FITC-Dextran in the apical layer (mg/mL). Since consistent concentrations and membrane surface areas were used for all experiments, the second term was simplified to a single constant C (cm/mg), yielding equation 2.

$${P}_{app} = C \frac{dQ}{dt} \qquad ( 2 )$$

Since Bruch's membrane mediates the selective transport between subretinal layers in vivo, the permeability of the hagfish membranes to molecules of various molecular weights after 7 days was determined (Table 3). Each membrane type had size-restrictive characteristics with an approximate 10-fold decrease in permeability for 10 and 70 kDa FITC-Dextran (Table 3). Transwells, as previously reported (Rickabaugh, E. et. al. ACS Biomat. Sci. Eng. 2023, 9(8), 5051), had the highest permeability.

A small decrease in permeability with UTaHM would be expected, similar to what is shown for the 70 kDA, but the difference with the small sugar (10 kDA) is much more.

TABLE 3
Permeability Coefficients (cm/s) of FITC-Dextran
across Bruch's Membrane and Transwell

Permeability Coefficient (cm/s)

| Membrane Type | 10 kDa (×10⁻⁷) | 70 kDa (×10⁻⁷) |
|---|---|---|
| aHM | 49.3 ± 12.0 | 3.38 ± 0.68 |
| TaHM | 40.5 ± 7.7 | 3.33 ± 0.43 |
| UTaHM | 9.3 ± 8.1 | 2.28 ± 1.90 |
| PE Transwell | 730 ± 369 | 98.3 ± 19.7 |
| (previously reported) | | |

### RPE Cell Culture

The cells used in this study were primary porcine RPE cells, isolated using a previously described method. (See Paterson, C. A., et. al. J. Vis. Exp. (207), e66079). Briefly, enucleated porcine eyes were cut along the ora serrata, and the anterior eye and vitreous were separated from the eye cup. The neural retina was then peeled back with tweezers and removed via vacuum aspiration. RPE cells were then detached from Bruch's membrane with trypsin and plated in a six-well plate, where they were grown in DMEM supplemented with 10% serum and 1% antibiotic/antimycotic. Media in these cultures was changed every other day, and once confluent, the serum content was reduced to 2% until experimental use. All cells used for experimentation were at passage 1.

To transfer cells onto hagfish membranes, cells were first removed from well plates with trypsin, centrifuged, and resuspended for 15 minutes in a 3 μg/mL solution of DNase in CaCl₂) to prevent clumping. The DNase solution was then removed, and cells were resuspended in DMEM with 10% serum and 1% antibiotic/antimycotic and seeded onto the apical face of hagfish membranes at a density of 3*10⁵ cells/cm². Culture media was changed twice a week. At the first media change post seeding, serum content was reduced to 1% throughout the remainder of the experimental period.

### Viability

Viability was measured using a 3-(4,5-Dimethylthiazol-2-yl)-2,5-Diphenyltetrazolium Bromide (MTT) assay. To perform the assay, an MTT (Millipore Sigma) stock solution of 5 mg/mL MTT in DPBS (−/−) was created. Prior to experimentation, the stock solution was diluted to 0.25 mg/mL in culture medium to create the MTT working solution. Culture medium was removed from pRPE cells and replaced with the MTT working solution (150 μL apically and 600 μL basally). Cells were incubated in the dark at 37 °C for 4 h. After incubation, cells were washed once with DPBS (−/−), which was then replaced with DMSO both apically and basally. Cells were incubated in the dark at room temperature for 5 min, then shaken in an orbital mixer at medium speed prior to analysis. Absorbance at 510 nm, which was determined to be the maximum absorbance in these samples, was measured in a Synergy HTX multi-mode reader (Biotek) with Gen5 software.

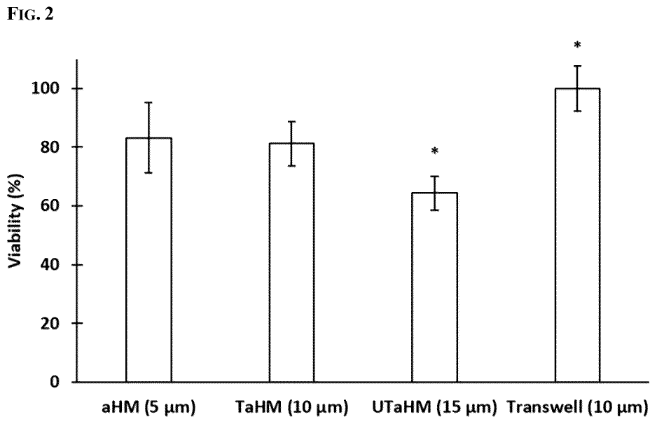

To quantify cellular viability prior to the formation of regions of cellular detachment on UTαHM, an MTT viability assay was performed. The calculated viability of cells cultured on αHM and TαHM was reduced to 83±12% and 81±8%, respectively, compared to a Transwell control (100%, FIG. 2). Cells cultured on UTαHM had the lowest viability of all experimental groups at 64±6%. These data were collected prior to the formation of any regions of detachment (FIG. 1), indicating that cell viability decreases on UTαHM prior to any visible markers of monolayer disruption.

### Lipid Staining

To visualize lipids found in the sub-RPE deposits, a LipidSpot™ (Biotium) lipid droplet stain was used. The stain was diluted in a 1:1000 ratio in PBS and added to cultures where RPE detachment occurred alongside deposits visible with Brightfield imaging. Samples were then incubated at 37 °C for 30 min and imaged with an LSM-710 (Zeiss) confocal microscope with an excitation wavelength of 488 nm.

### TEER

Transepithelial electrical resistance (TEER) was used to assess RPE monolayers for polarization and overall monolayer integrity as an in vitro oBRB. Prior to recording resistance, the media was changed, and the plate was placed at room temperature for 15 minutes to stabilize plate temperature. After the plate had cooled, cell culture inserts were removed from the well plate and placed in a placed in a ENDOHM-24snap in an EVOM2 from World Precision Instruments. Resistance values were noted once the measurement had stabilized, and values were presented in units of Ω*cm², with 0.33 cm² as the culture surface area. Each insert was measured twice to ensure device consistency. TEER was performed throughout a 3-week culture period, with measurements at 7, 10, 14, 17, and 21 days post-seeding. Imaging was performed for up to three weeks to assess monolayer health (FIG. 1).

After collecting preliminary data, the culture period was extended out to 5 weeks with measurements taken twice per week. Between 3 and 5 weeks of culture on UTαHMs, RPE cells began to die and detach from the membrane (FIG. 1A), while cells cultured on αHMs retained a consistent monolayer and their characteristic cobblestone morphology (FIG. 1). Within 7 days of seeding, cultures grown on both UTαHMs and TαHMs formed domes that extended apically to the rest of the monolayer (FIGS. 1C and 1D). Domes consistently disappeared by 14 days post-seeding, but regions where they had formed corresponded with regions of cell death at later time points.

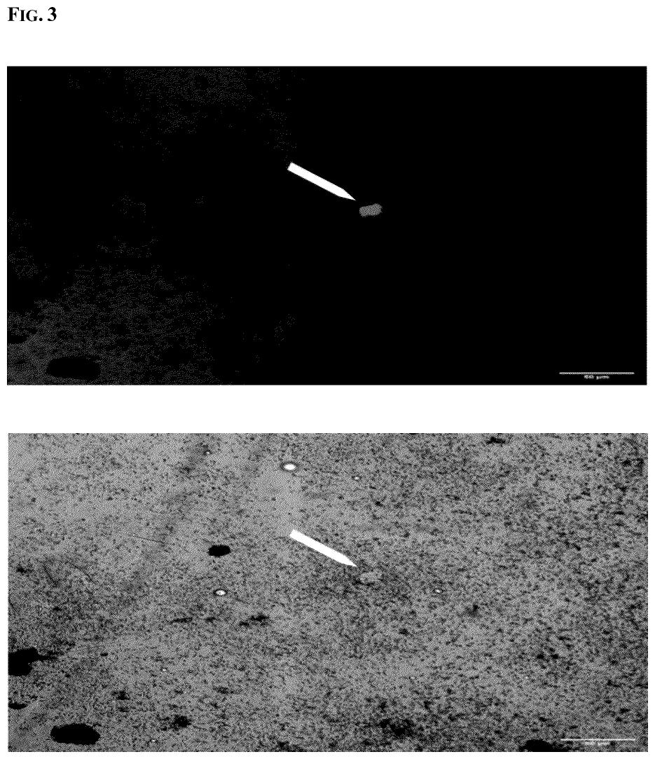

In addition to patches of cell death, cultures on TαHMs and UTαHMs formed deposits beneath the RPE monolayer. After up to three months of culture, the cell layer eventually detached entirely, leaving deposits attached to the membrane (FIG. 3, right). These deposits showed high lipid content (FIG. 3, left). Further analysis confirmed the presence of Apolipoprotein E (ApoE), marking a comparison between these deposits and drusen deposits found in AMD. The presence of drusenoid deposits formed by cells on TαHM and UTαHM support hagfish membranes as a mimetic model of aging and indicate that Bruch's membrane plays a role in drusen formation.

### Example 2: Analysis of RPE Monolayer Disruption

### ICC

Immunocytochemistry (ICC) was performed on cultures after 21 days of growth to visualize the relationship between zonula occludens-1 (ZO-1) and apolipoprotein E (ApoE). Cells were analyzed using an LSM-710 (Zeiss) confocal microscope and excitation wavelengths of 405, 488, and 633 nm for nuclear, ApoE, and ZO-1 imaging, respectively. Maximum intensity projections of an 8-slice Z-stack were also obtained for each sample.

In preparation for staining, each sample was fixed in a 4% paraformaldehyde solution for 10 minutes after 21 days of culture. After fixation, cells were permeabilized with a 1% Triton-X solution for 10 minutes and washed 3 times with DPBS for 5 minutes each. Cells were then blocked in a 3% bovine serum albumin (BSA) solution for 1 hour. After blocking, primary ZO-1 antibodies and primary ApoE antibodies were added at 1 μg/ml and 3 μg/ml, respectively, and incubated at 4 °C overnight. Once the antibodies had incubated, the samples were washed 3 times with DPBS, and a secondary conjugated antibody was added at 4 μg/ml for 1 hour. Cells were then washed again and counterstained with a NucBlue live ReadyProbes reagent which was added according to manufacturer's instructions. After a final set of washes, membranes were removed from the plastic culture inserts and secured on glass microscope slides using clear nitrocellulose. Samples were cured overnight at room temperature before imaging.

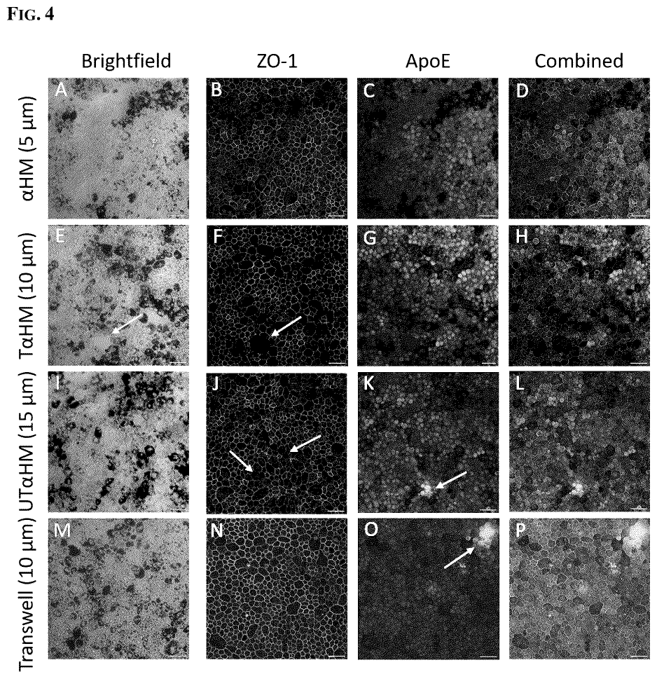

ICC staining showed the formation of ApoE deposits and the degradation of the RPE monolayer on cultures of each membrane type after 21 days of growth (FIG. 4). The ICC imaging revealed differences between cells cultured on various hagfish membranes. Cells on αHMs showed a healthy morphology that corresponded to consistent expression of both ApoE and ZO-1 (FIG. 4, A-D). Conversely, cells cultured on TαHMs developed gaps in their monolayer that was also seen in disrupted ZO-1 expression (FIG. 4, E-G). While UTαHM cultures experienced cell death starting around 3 weeks, prior to that point, they seemed to exhibit normal morphology in Brightfield imaging (FIG. 4I). At that timepoint, however, they also experienced an increase in ApoE expression that corresponded with a nearby delocalization of ZO-1 (FIG. 4I-L). Transwell controls were similar to αHMs with a consistent monolayer and ZO-1 expression but showed a localization of ApoE similar to that of UTαHM cultures. This localization, however, did not accompany any disruption of ZO-1.

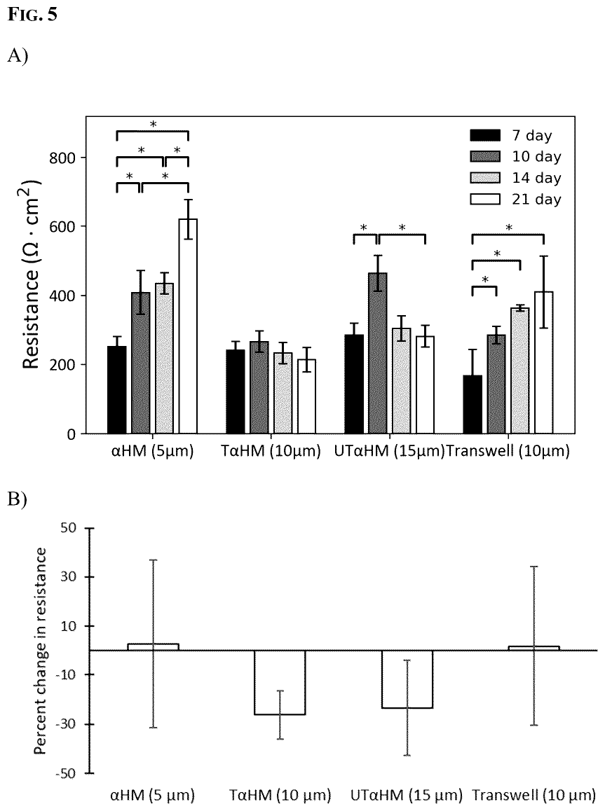

Changes in ZO-1 expression suggested that monolayer integrity may be impacted prior to visible changes in cell morphology. To verify monolayer barrier function quantitatively, TEER was performed to measure resistance across the culture at time points between 7 and 21 days (FIG. 5, A). Cells were also cultured for up to 5 weeks to evaluate whether long-term differences between cells on each membrane type persisted. TEER was measured from these cultures twice per week, beginning at 10 days post-seeding. Once the monolayer matured (reaching 300 Ω*cm²), the first reading greater than 300 Ω*cm² was used as a reference value for that sample. Total change in resistance, calculated as a percent change from the first measurement greater than 300 Ω*cm², was also recorded (FIG. 5, B).

Over the 21-day culture period, TEER measurements on cell layers grown on αHMs and Transwells increased in resistance value, indicating an increase in monolayer integrity over that time. TαHMs experienced no significant changes throughout that time but also never reached what is typically considered a mature resistance (300 Ω*cm²). Conversely, cells growing on UtαHMs had an initial increase in resistance from 7 to 10 days and a subsequent decrease from 10 to 14 days. When cultured for longer periods, cells on both αHMs and Transwells remained stable after reaching a mature monolayer, while cells on TαHM and UTαHM experienced a decrease in resistance value. Additionally, 25% of TαHM cultures experienced a decrease in resistance within 14 days of forming a stable monolayer, while 50% of UTαHM cultures decreased within that same timeframe. These changes in resistance on TαHMs and UTαHMs correspond to the visual disruption of ZO-1 seen in FIG. 4. These findings further support that thickened membranes impede healthy monolayer formation in RPE cell culture.

### RPE Morphology and Junctional Analysis

The programs ImageJ and CellProfiler were used to assess cell morphology and junction length after 3 weeks of culture on the different membrane types. After performing ICC, images of the same size and exposure time were imported into each program as TIF files. To measure average junction length, the “Ridge detection” plugin in ImageJ was used. Form factor and eccentricity, measures of deviation from a perfect circle and the eccentricity of an ellipse, respectively, were measured using the “neighboring cells” pipeline to determine consistent RPE morphology. The number of adjacent cells was measured using the “MeasureObjectSizeShape” pipeline to ensure RPE cells had an appropriate hexagonal morphology, where the ideal number of adjacent cells would be 6.

After 3 weeks of cell culture on the various membrane types, images of pRPE cells were analyzed with ImageJ and CellProfiler to calculate average junction length, form factor, eccentricity, and the number of adjacent cells (Table 4). Notably, none of these analyses yielded significant differences between cells cultured on any of the membrane types. These results indicate that the thickness of Bruch's membrane alone did not affect any of these morphological measures within the culture period.

TABLE 4
Membrane type alone did not influence
RPE morphology or junction length

| | | | | Average |
|---|---|---|---|---|
| Membrane | | | Number of | junction |
| type | Form factor | Eccentricity | adjacent cells | length (μm) |
| αHM | 0.76 ± 0.07 | 0.63 ± 0.15 | 5.2 ± 1.5 | 11.4 ± 6.2 |
| TαHM | 0.77 ± 0.06 | 0.63 ± 0.15 | 5.3 ± 1.2 | 10.4 ± 4.4 |
| UTαHM | 0.75 ± 0.06 | 0.64 ± 0.16 | 5.4 ± 1.2 | 11.0 ± 5.7 |
| Transwell | 0.77 ± 0.06 | 0.63 ± 0.14 | 5.4 ± 1.3 | 10.0 ± 4.2 |

### ELISA

During each media change, spent media was collected and frozen at −20 °C for protein secretion analysis. For this procedure, samples were taken from cells 14, 17, and 21 days post-seeding, which were timepoints chosen based on preliminary TEER results. Protein analysis was performed using a Q-plex Human Angiogenesis (9-plex) ELISA kit (Quansys). The ELISA protocol was performed according to manufacturer instructions, with a 1:2 dilution of sample to buffer and imaging performed on a Q-view LS imager.

ELISA was used to show the connection between changes in Bruch's membrane and progression to wet AMD. ELISA was done by measuring pro-angiogenic secretions after 3 weeks of culture.

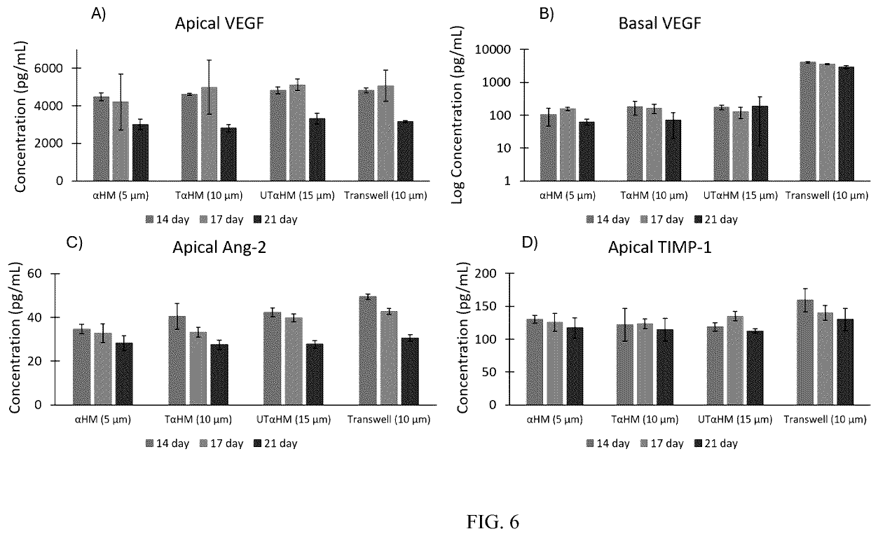

From 14 to 21 days of cell culture, there were few differences between membrane types. VEGF (both apically and basally) and Ang-2 (apical) behavior followed similar trends of decreasing throughout the culture period (FIG. 6). Apical TIMP-1 secretion, however, had a statistically significant increase from 14 to 17 days on UTαHM, followed by a decrease from 17 to 21 days (FIG. 6). The spike in TIMP-1 secretion aligned with the timing of ApoE aggregation (FIG. 4) and monolayer breakdown (FIG. 5), suggesting a relationship between TIMP-1, ApoE, and the progression of AMD.

In addition to measuring VEGF and Ang-2 secretions, TIMP-1 secretion was also measured. Cells on UTαHM had increased secretion of TIMP-1 at 17 days of growth, followed by a decrease at 21 days. The decrease in VEGF and Ang-2 suggest that while Bruch's membrane plays a role in the progression of AMD, it alone does not lead to angiogenesis in late-AMD. This result resembles other studies that investigated early AMD where VEGF and Ang-2 expression is more affected after chronic stress and high levels of cell death. These studies align with the current practice of delivering anti-angiogenic treatments to AMD patients only after cell death has occurred in the macula. The spike in TIMP-1 after 17 days indicates that the progression of AMD caused by Bruch's membrane is more closely related to this protein than VEGF and Ang-2, the targets of most AMD treatments. Normally, TIMP-1 works with other TIMP proteins and MMPs to regulate the ECM and Bruch's membrane (see Krogh Nielsen M, et. al. Acta Ophthalmol (Copenh). 97:84-90 (2019)). This function could explain why alterations to Bruch's membrane cause inconsistent protein expression. Additionally, TIMP-1 expression is increased in AMD patients with geographic atrophy (GA) and it has been described as both angiogenic and anti-angiogenic. Increased levels of TIMP-1 responding to changes in Bruch's membrane implies that TIMP-1 may be a key player in the transition from early to late-stage AMD.

### Statistical Analysis

SAS studios was used for all statistical analyses in this study. Numerical values consisted of at least 3 biological replicates, where each individual replicate was comprised of two technical replicates averaged together. Average values and standard deviation were calculated based on calculated values for the biological replicates. These values were analyzed with two-way ANOVA with a set a level of 0.05 to assess for the effect of culture time and membrane thickness on cell outputs, and to account for interaction effects between the variables. In samples where only one time point was analyzed, a one-way ANOVA with a set a level of 0.05 was used. Tukey post-hoc analysis was performed to assess pairwise statistical significance.

The present disclosure enables one of skill to make and use systems and methods provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary and not limiting thereof. The scope of the invention is, therefore, set out in the appended claims.

Although specific embodiments of the present disclosure are illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

1. A composition comprising:
(a) a membrane for supporting growth of cells, the membrane being made of hagfish intermediate filament α wherein the membrane has a minimum thickness of 12 μm; and
(b) a layer of retinal pigment epithelial (RPE) cells, or retinal pigment epithelial cell precursors, on the membrane.

2. The composition of claim 1, wherein the RPE cells are primary RPE cells.

3. The composition of claim 1, wherein the layer of RPE cells is a monolayer.

4. The composition of claim 1, wherein the layer of RPE cells is at least a density of 3×10⁵ cells/cm².

5. A synthetic membrane system, comprising:
a membrane having:
a side facilitating the growth of a monolayer of cells;
a thickness greater than 12 μm; and
the membrane comprising hagfish intermediate filament α.

6. The system of claim 5, further comprising:
a cell culture insert on which the membrane sits; and
a silicone ring holding the membrane to the cell culture insert.

7. The system of claim 5, wherein the membrane is circular with a diameter of between about 10 mm to about 20 mm.

8. A method of making a Bruch's membrane model comprising:
(i) spreading a solution of hagfish intermediate filament α onto a disk to form a membrane at a concentration of 1% w/v of hagfish intermediate filament α;
(ii) drying the membrane;
(iii) removing the dried membrane from the disk;
(iv) rehydrating the membrane;
(v) adding the dried membrane to a cell culture insert; and
(vi) seeding the membrane-cell culture insert with cells.

9. The method of claim 8 further comprising, growing the cells in a monolayer.

10. The method of claim 8, wherein the cells are retinal pigment epithelial (RPE) cells.

11. The method of claim 8, wherein the RPE cells are primary porcine RPE cells.

12. The method of claim 8, wherein the RPE cells are seeded at a density of 3×10⁵ cells/cm².

13. A synthetic membrane system made by the methods of claim 8.

14. A method of identifying compositions for treating or lessening the severity of age-related macular degeneration in a subject comprising:
(i) administering a test composition to the Bruch's membrane model made by claim 8; and
(ii) measuring the effect on the Bruch's membrane model;
wherein the composition is selected as a candidate on the basis of treating or lessening the severity of age-related macular degeneration.

15. The method of claim 14, wherein the treating or lessening the severity of age-related macular degeneration comprises decreasing the thickness, decreasing the stiffness, increasing the permeability, or increasing the elasticity of the Bruch's membrane.
